(19) United States
(12) Patent Application Publication
(10) Pub. No.: US 2009/0032030 A1
(43) Pub. Date: Feb. 5, 2009

(54) APPARATUS FOR TREATMENT OF SLEEP APNEA

(76) Inventor: R. Sam Callender, Golden, CO (US)

Correspondence Address: DORR, CARSON & BIRNEY, P.C.;ONE CHERRY CENTER, 501 SOUTH CHERRY STREET, SUITE 800, DENVER, CO, 80246, US

(21) Appl. No.: 12/016,361

(22) Filed: Jan. 18, 2008

Related U.S. Application Data
(63) Continuation-in-part of application No. 11/830,335, filed on Jul. 30, 2007.

(51) Int. Cl. A61F 5/00

(52) U.S. Cl. 128/845

(57) ABSTRACT

A dental aligner can be placed between the patient's upper and lower teeth to help maintain proper positioning of the mandible for treatment of sleep apnea and sleep disorders. The aligner has an upper section to receive the upper teeth, a lower section to receive the lower teeth, and an adjustment mechanism to removably secure the upper section to the lower section and provide a degree of adjustability along an anterior-posterior axis. The upper and lower sections of the aligner have polymeric bodies that extend on the occlusal and labial-buccal aspects, but not on the lingual aspects of the patient's teeth. Labial-buccal archwires are embedded in the polymeric bodies of the upper and lower sections of the aligner for structural reinforcement. Bone screws or buttons on the upper and lower sections of the aligner can provide attachment points for elastics to assist in moving the mandible forward.

### RELATED APPLICATION

The present application is a continuation-in-part of the Applicant's U.S. patent application Ser. No. 11/830,335, entitled “Method And Apparatus For Treatment Of Sleep Apnea,” filed on Jul. 30, 2007.

### BACKGROUND OF THE INVENTION

1. Field of the Invention

The present invention relates generally to the field of devices for treatment of sleep apnea and related sleep disorders. More specifically, the present invention discloses an aligner for treatment of sleep apnea and related sleep disorders by positioning the mandible forward relative to the maxilla.

2. Statement of the Problem

Sleep disorders, such as sleep apnea, snoring and bruxism, can have potentially serious health and social consequences, including daytime fatigue, a compromised immune system, poor mental and emotional health, irritability and lack of productivity. These sleep disorders have also been linked to an increased risk of diabetes, high blood pressure, stroke and heart attacks. Snoring and sleep apnea are both generally caused by blockage of the pharyngeal airway by excess tissue when the muscles associated with the tongue, pharynx, mandible and soft palate relax during sleep. As the tongue relaxes, it tends to move posteriorly and can block the airway. Snoring is often caused by partial obstruction of breathing during sleep. In contrast, sleep apnea occurs when the tongue and soft palate collapse posteriorly and completely block the airway.

Many approaches have been tried in the past to treat sleep apnea and snoring. Various types of surgery, such a uvulapharyngoplasty and other types of surgery of the soft palate, oropharynx and nasopharynx have using in treating these conditions. However, any type of invasive surgery has obvious risks and disadvantages.

The prior art in this field also includes a variety of intra-oral dental appliances and mandibular advancement devices, such as disclosed in U.S. Patent Application Publication No. 2007/0079833 (Lamberg), U.S. Pat. Nos. 5,365,945 and 6,729,335 (Halstrom) and others. These devices typically employ one or more polymeric dental appliances (e.g., bite trays, retainers, or splints) that fit over or contact a patient's teeth to shift the mandible forward relative to the maxilla to keep the airway open during sleep. However, since the forces used to reposition the mandible are carried by the teeth, these forces can also cause undesired repositioning of the teeth as well. In addition, many conventional dental appliances are relatively bulky and obtrusive, which interferes with the patient's ability to sleep and can result in poor patient compliance.

U.S. Pat. No. 6,109,265 (Frantz et al.) discloses a dental appliance with upper and lower plastic trays that conform to the patient's upper and lower teeth, soft tissue and palate. Elastic bands extend between pairs of retention hooks on the upper and lower trays to pull the mandible forward. Here again, the forces used to reposition the mandible are largely carried by the teeth.

U.S. Pat. No. 6,983,752 (Garabadian) discloses another example of a dental appliance with upper and lower trays for treatment of sleep disorders. Bite pads attached to the upper and lower trays allow limited vertical and lateral movement, while maintaining the occlusal surfaces of the trays in a predetermined spaced relationship. A number of buttons are attached to the buccal surfaces of the trays to attach elastic bands extended between the upper and lower trays.

U.S. Pat. No. 7,216,648 (Nelson et al.) discloses an intraoral appliance that includes a posterior extension supporting and stabilizing the soft tissue of the patient's palate to treat snoring and sleep apnea.

U.S. Patent Application Publication Nos. 2007/0006884 and 2004/0177852 (Abramson) disclose a dental appliance that includes an upwardly-extending intra-oral nasal dilator supported by wires from a base unit that fits over the patient's lower teeth. The nasal dilator has two acrylic nasio-labial buttons.

Herbst appliances are commonly used in orthodontics to reposition the mandible in a more forward position to treat over-bite conditions. An example of a Herbst appliance is disclosed in U.S. Patent Application Publication No. 2006/0234180 (Huge et al.). A Herbst mechanism typically spans between the upper posterior teeth and the lower canine region. One common configuration uses a two-part telescoping mechanism consisting of a rod connected to the patient's lower arch and a tube connected to the upper arch. The ends of these telescoping segments have eyelets engaging pivots secured to orthodontic bands on the patient upper and lower arches. As the patient closes his or her teeth, the telescoping mechanism slides together until a predetermined limit is reached. Beyond that limit, the telescoping segments exert a force that tends to reposition the mandible forward with respect to the maxilla. Here again, the forces for repositioning the mandible are carried by the patient's teeth, and can undesirably change the positions of the patient's teeth as well

The prior art in the fields of orthodontics and intra-oral appliances for treatment of snoring and sleep disorders includes a number of two-piece aligners or positioners. These devices typically include an upper section to receive the patient's upper teeth, a lower section to receive the patient's lower teeth, and some means for removably holding the upper and lower sections together (e.g. elastics or hook-and-loop fasteners). Examples include U.S. Pat. Nos. 5,642,737 (Parks), 5,884,628 (Hilsen), 5,611,355 (Hilsen), 6,450,167 (David et al.) and 4,505,672 (Kurz).

U.S. Pat. No. 6,129,084 (Bergersen) discloses another example of an intra-oral appliance to prevent snoring. The device has upper and lower U-shaped plates that are joined to form a hinge. The upper plate has a labial-buccal wall but no lingual wall, which facilitates anterior positioning of the tongue.

Thus, most conventional aligners and positioners extend over the lingual, labial-buccal and occlusal aspects of the patient's teeth. This tends to result in relatively large, bulky appliances that restrict the range of motion of the patient's tongue. Those appliances, such as the Bergersen device, which attempt to address this problem by omitting some lingual portions of the appliance, have had to compensate by increasing the bulk of other portions of the appliance to provide structural support.

Solution to the Problem. The present invention addresses the shortcomings associated the prior art in this field by providing an aligner that extends only on the buccal-labial and occlusal aspects of the patient's teeth. This enables the aligner to be made much smaller, lighter and less obtrusive. Structural support and rigidity is provided by labial-buccal archwires embedded in the polymeric bodies of the upper and lower sections of the aligner, along with a series of ball clasps extending interproximally between the posterior teeth for retention. Optionally, the aligner can be used in conjunction with bone screws connected by elastics to transmit the forces used to reposition the mandible directly to the bone structures of the mandible and maxilla, with only incidental forces being carried by the teeth.

### SUMMARY OF THE INVENTION

This invention provides an aligner that can be placed between the patient's upper and lower teeth to help maintain proper positioning of the mandible in a forward position for treatment of sleep apnea and snoring. The aligner has an upper section to receive the upper teeth, a lower section to receive the lower teeth, and an adjustment mechanism to removably secure the upper section to the lower section and provide a degree of adjustability along an anterior-posterior axis. The upper and lower sections of the aligner have polymeric bodies that extend on the occlusal and labial-buccal aspects, but not on the lingual aspects of the patient's teeth. Labial-buccal archwires are embedded in the polymeric bodies of the upper and lower sections of the aligner for structural reinforcement. A number of ball clasps can extend interproximally between the posterior teeth for retention. Optionally, the upper section of the aligner can include a posterior extension to lift and tighten the soft palate.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is a side perspective view of a patient's dental anatomy showing upper and lower bone screws 20 and 22 connected by an elastic 25, and also showing an aligner 30.

FIG. 2 is a perspective view of a bone screw

FIG. 3 is an exploded perspective view of the upper and lower sections 31, 32 of an aligner 30.

FIG. 4 is a vertical cross-sectional view of an aligner 30 with upper and lower molars.

FIG. 5 is a top view of the lower section 32 of an aligner 30 on a patient's lower teeth.

FIG. 6 is a side view of an embodiment of an aligner 30 with a screw adjustment mechanism allowing a range of anterior-posterior movement between the upper and lower sections 31, 32 of the aligner 30.

FIG. 7 is a side view of an embodiment of an aligner 30 with another type of screw adjustment mechanism.

FIG. 8 is a side view of a patient's dental anatomy with multiple lower bone screws.

FIG. 9 is a side view of a patient's dental anatomy showing another arrangement for attaching an elastic 25 between a maxillary bone screw 20 and multiple mandibular bone screws 22a-22e.

FIG. 10 is a side view of a patient's dental anatomy showing another arrangement for attaching an elastic 25 between multiple maxillary bone screws 20a-20e and mandibular bone screws 22a-22e.

FIG. 11 is an exploded perspective view of the upper and lower sections 31, 32 of another embodiment of the aligner 30 with buttons 41, 42 formed in the plastic of the upper and lower sections for retaining elastics.

FIG. 12 is a top view of the lower section 32 of the aligner 30 illustrated in FIG. 11 on a patient's lower teeth.

FIG. 13 is a side view of the embodiment of the aligner 30 illustrated in FIG. 11 with a screw adjustment mechanism allowing a range of anterior-posterior movement between the upper and lower sections 31, 32 of the aligner 30, with elastics 25 extending between the upper and lower sections.

FIG. 14 is a bottom view of an embodiment of the upper section 31 of an aligner 30 incorporating a posterior extension 70 to elevate and tighten the patient's soft palate tissue 17.

FIG. 15 is a side cross-sectional view of the embodiment of the aligner 30 illustrated in FIG. 14 showing the manner in which the soft tissue 17 of the patient's soft palate is elevated by the posterior extension 70 of the upper section 31 of the aligner 30.

FIG. 16 is a vertical cross-sectional view showing another embodiment of an aligner that includes a screw 80 for adjusting the minimum vertical separation between the upper and lower sections 31, 32 of the aligner. The screw 80 is threaded into a nut 85 embedded into the occlusal surface of the upper section 31 of the aligner.

FIG. 17 is an orthogonal vertical cross-sectional view of the embodiment depicted in FIG. 16.

### DETAILED DESCRIPTION OF THE INVENTION

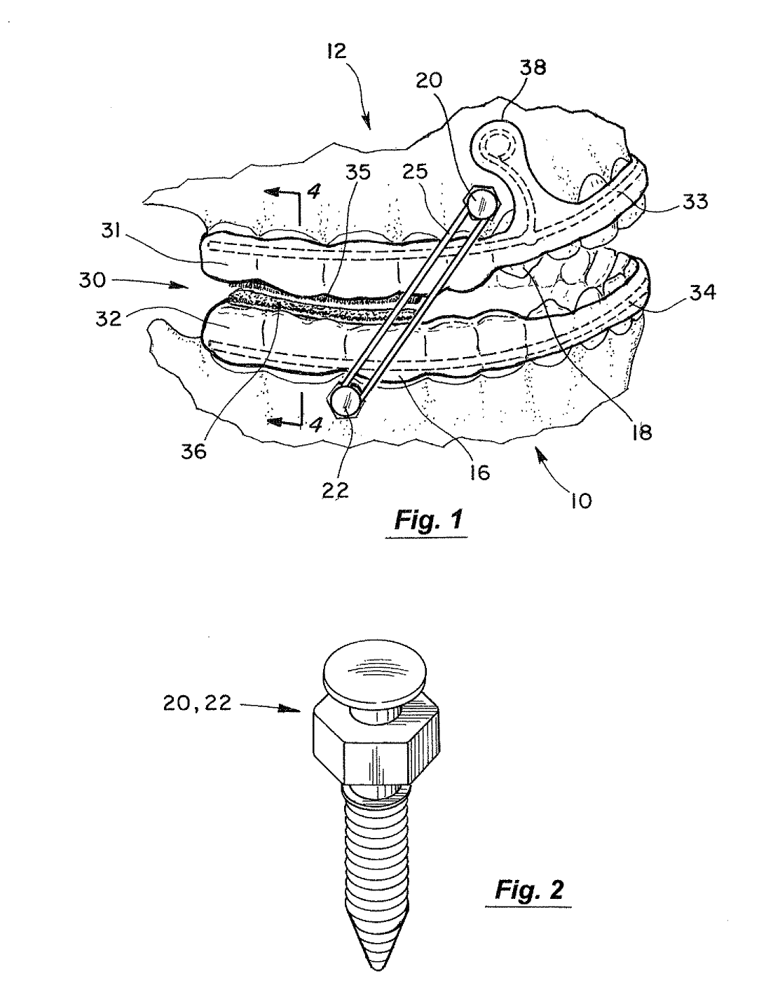

Turning to FIG. 1, a side perspective view of a patient's dental anatomy is provided showing the major components of one embodiment of the present invention. A maxillary bone screw 20 is attached to the basal bone of the anterior portion of the maxilla 12. For example, maxillary bone screws 20 can be attached above and posterior to the upper cuspids 18 or bicuspids above the crown and between the teeth at the mucosal-gingival junction as shown in FIG. 1. The maxillary bone screws 20 can also be implanted behind and above the patient's molars. Similarly, mandibular screws 22 are implanted in the basal bone structure of the posterior portion of the mandible 10, typically below and between the first and second molars 16. However, the mandibular screws 22 could be implanted below any of the patient's lower molars, bicuspids, cuspids, or other posterior teeth. One pair of maxillary and mandibular bone screws 20, 22 would usually be attached on the left side of the patient and a second pair of bone screws would be attached on the right side for left-right symmetry.

A number of bone screws are commercially available and have long been used, for example, for orthodontic traction for tooth movement. FIG. 2 is a perspective view of one type of bone screw. Most conventional bone screws include a screw portion that can be threaded into the bone, and a head portion extending beyond the soft tissue covering the bone that can be rotated with a dental tool. Optionally, the head of each bone screw 20, 22 can include an annular recess to hold an elastic 25. The threaded portions of the bone screws can be implant into the basal bone structures of the maxilla 12 and mandible 10 using conventional techniques. A small initial incision is often made through the overlaying soft tissue. Holes for the bone screws can be drilled and tapped in the bone, although many bone screws are self-drilling and self-tapping.

After installation, the head of each bone screw typically extends outward in the buccal direction beyond the surface of the soft tissue covering the bone so that elastics to be easily attached to the exposed heads of the bone screws. Alternatively, the bone screws could be attached on the lingual side of the dental arches with the heads of the bone screws extending in the lingual direction, although this arrangement may have the disadvantage of crowding the tongue 14. After installation of the bone screws, elastics 25 are stretched between the pairs of maxillary and mandibular bone screws 20, 22 to exert forces that tend to move the mandible 10 forward and upward with respect to the maxilla 12. It should be noted that the major forces of repositioning the jaw are carried by the bone structures of the maxilla 12 and mandible 10, rather than the teeth.

For example, conventional orthodontic elastic bands 25 (e.g., class 2 bands) can be used for this purpose. It should be noted that a progressive series of bands of different mechanical properties can be used over time. These elastics 25 can be easily attached to the bone screws 20, 22 by the patient before going to sleep and then removed after waking. Multiple elastics can also be attached between pairs of maxillary and mandibular bone screws 20, 25, if desired. Other types of elastic members could be substituted.

An aligner 30 is placed between the patient's upper and lower teeth to ensure proper positioning of the mandible 10 with respect to the maxilla 12. The body of the aligner 30 can be made of a polymeric material (e.g., acrylic) using conventional orthodontic techniques. The upper and lower surfaces of the aligner 30 incorporate a series of recesses to receive the patient's upper and lower teeth. Since only nominal forces are carried by the aligner 30 and teeth, the aligner 30 can have a very light construction with a minimal thickness sufficient to contact the cusps of the teeth.

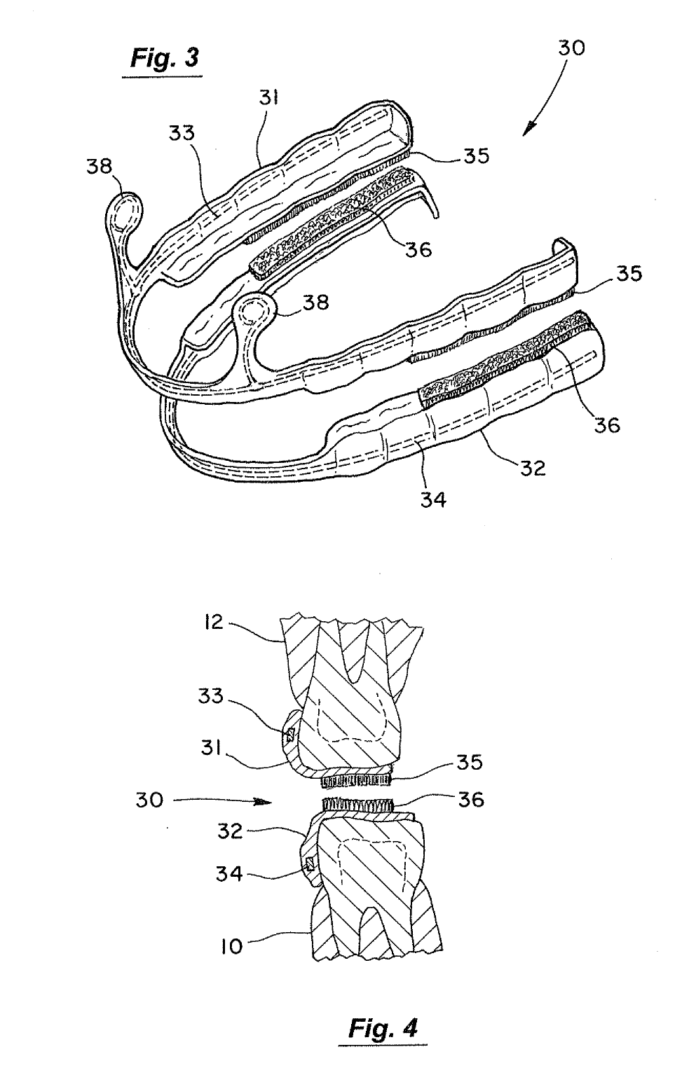

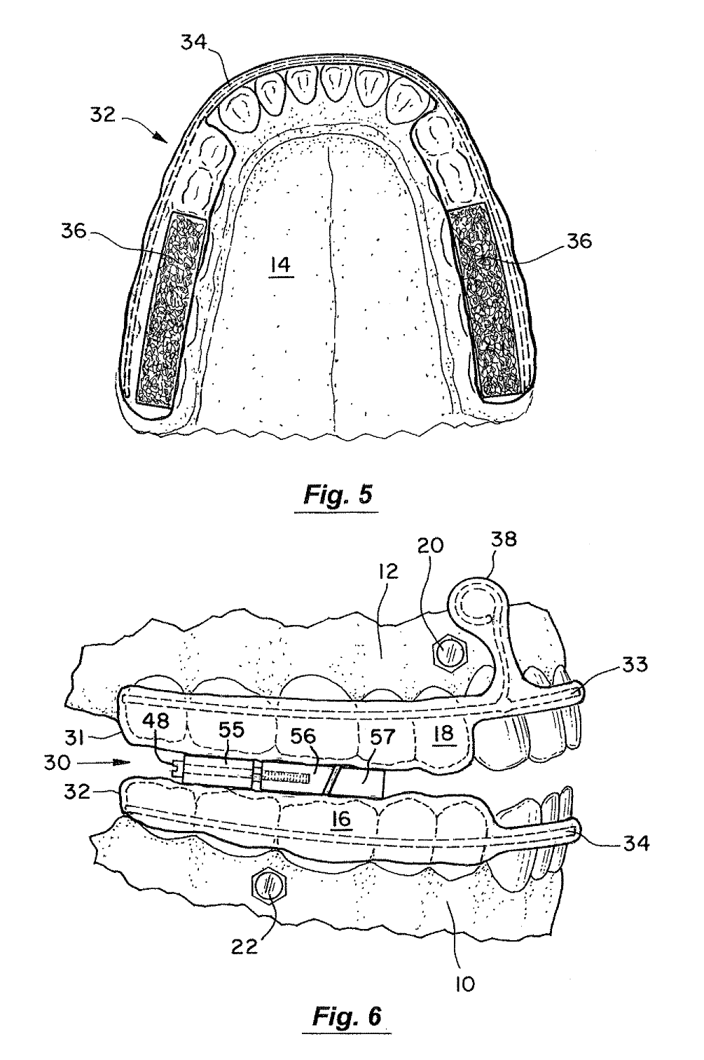

The embodiment of the aligner 30 shown in the exploded perspective view illustrated in FIG. 3 has a two-piece construction that includes an upper section 31 and a lower section 32, with an adjustment mechanism to provide a degree of adjustability along an anterior-posterior axis between the upper and lower sections 31, 32. This in turn provides a degree of adjustability between the dental arches. The upper section 31 of the aligner has a polymeric body with a series of recesses intended to receive the patient's upper teeth, while the lower section 32 has a polymeric body with a series of recesses to receive the patient's lower teeth. FIG. 5 is a top view of the lower section 32 of an aligner 30 on a patient's lower teeth. It should be understood that the upper and lower sections 31, 32 are separate in this embodiment, but work together to function as a single aligner to maintain a desired positioning between the patient's maxilla and mandible.

The aligner 30 includes upper and lower labial-buccal archwires 33, 34 embedded in the polymeric bodies of the upper and lower sections 31, 32 of the aligners 30 for increased strength and rigidity. It should be understood that the term “archwire” should be broadly construed to cover wires, multi-strand cables, bands or elongated members of any type. Additional mesh or reinforcing members can also be embedded in the aligner 30 for added strength. In the embodiment shown in the drawings, the anterior portion of the aligner 30 includes a thin layer of acrylic material covering the upper and lower archwires 33, 34 adjacent to the labial surfaces of the anterior teeth to prevent irritation of the patient's lips by the archwires 33, 34. These labial archwires 33, 34 can also serve an orthodontic function by preventing the incisors from erupting.

The aligner 30 extends primarily on the labial-buccal and occlusal aspects of the teeth, as shown in the vertical cross-sectional view depicted in FIG. 4. Note that the upper and lower sections 31, 32 of the aligner 30 do not extend beyond the lingual cusps of the molars to the lingual aspects of the teeth. This essentially eliminates intrusion of the aligner 30 on the lingual aspect of the dental arch, reduces infringement on tongue space, and thereby enhances patient comfort. The polymeric bodies of the upper and lower sections 31, 32 can be limited to the posterior teeth to further reduce the bulk of the aligner 30. Modifications to the shape, contour and position of the plastic portions of the aligner can be made to stimulate the tongue as needed for function.

In the embodiment of the aligner 30 shown in FIG. 3, the occlusal surfaces of the upper and lower sections 31, 32 of the aligner 30 bear complementary patches 35, 36 of a hook-and-loop fastener material (e.g., Velcro® material) that removably secure the upper and lower sections 31, 32 together. FIG. 4 is a corresponding vertical cross-sectional view of the aligner 30 including upper and lower molars Over the course of treatment, the healthcare professional can separate the fastener patches 35, 36 and adjust the anterior-posterior positioning the upper and lower sections 31, 32 of the aligner 30 to accommodate changes in the patient's jaw position. Optionally, a numbers of lines or other visual indicia can be placed on the upper and lower sections 31, 32 of the aligner 30 to assist the healthcare provider in measuring how much adjustment has been made over the course of treatment. In particular, marks can be made on the side of the aligner to indicate how much adjustment has been made.

It should be understood that other types of adjustment mechanisms could be substituted to adjust the anterior-posterior positions of the upper and lower sections 31, 32 of the aligner 30. For example, FIG. 6 is a side view of an embodiment of an aligner 30 with a screw adjustment mechanism allowing a range of anterior-posterior movement. A first block 55 is secured to the occlusal surface of the upper section 31 of the aligner, and a second block 57 is secured to the occlusal surface of the lower section 32 of the aligner 30. An adjustment screw 48 extends through the first block 55 and is threaded into a third block 56 having an angled anterior face. The head of the screw 48 remains accessible on the posterior face of the first block 45. This enables the healthcare provider to adjust the spacing between the blocks 55, 56 by using a small tool to turn the head of the adjustment screw 48. The anterior face of the third block 56 contacts the complementary angled posterior face of the second block 57 attached to the lower section 32 of the aligner 30, to thereby guide the upper and lower sections 31, 32 of the aligner 30 into a desired anterior-posterior relationship when the jaw is closed. This configuration allows the patient a range of motion in opening and closing the jaw because the angled surfaces of the second and third blocks 57 and 56 are separate and free to move with respect to one another. The blocks 55, 56 and 57 also serve as bite blocks to maintain a desired vertical separation between the patient's upper and lower teeth while the aligner 30 is in place. Optionally, a number of reinforcing pins can be placed in sliding engagement with the blocks 55, 56 parallel to the axis of the adjustment screw 48 for additional support.

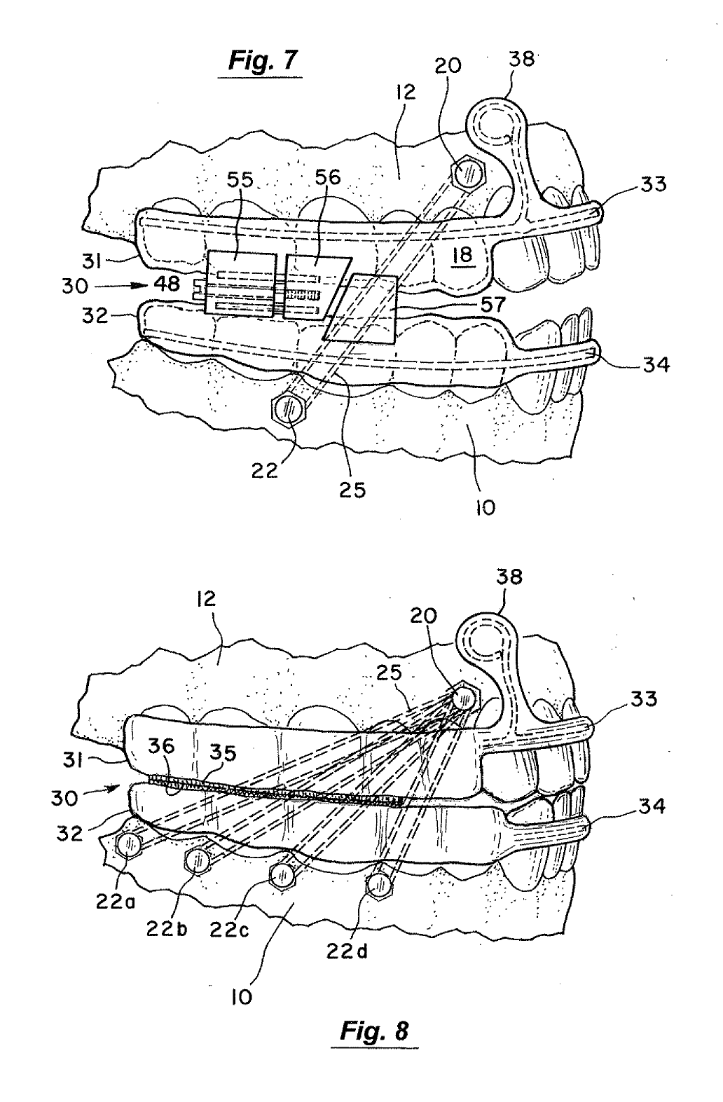

FIG. 7 is a side view of another embodiment of an aligner 30 with a screw adjustment mechanism employing bite blocks 55, 56 and 57 with angled surfaces that allow the patient to open and close the jaw. The first block 55 and second block 57 are attached to the buccal aspects of the upper and lower sections 31, 32, respectively, of the aligner 30. Here again, an adjustment screw 48 enables the healthcare provider to adjust the anterior-posterior spacing between blocks 55 and 56. The complementary angled surfaces on blocks 56 and 57 guide the upper and lower sections 31, 32 of the aligner 30 into the desired anterior-posterior relationship when the jaw is closed.

As previously noted, a primary advantage of the present invention is that the forces used to reposition the jaw are carried by the bone screws attached to the maxilla and mandible, rather than be carried by the teeth. However, it should be understood that the present invention provides an additional advantage in that the aligner 30 can be made lighter and thinner due to the minimal forces that it carries. For example, the aligner 30 can be configured to primarily engage the patient's posterior teeth (i.e., molars and bicuspids).

Optionally, the anterior portions of the upper and lower sections 31, 32 of the aligner 30 can be reduced in size or replaced with labial archwires 33, 34. As shown in the drawings, the upper archwire 33 can have a posterior portion embedded within the polymeric body of the upper section 31 and an anterior portion that extends around the labial aspect of the upper anterior teeth. Similarly, the lower archwire 34 can have a posterior portion embedded within the polymeric body of the lower section 32 and an anterior portion that extends around the labial aspect of the patient's lower anterior teeth.

Optionally, an extension or shield 38 can extend upward on the labial aspect of the maxillary anterior portion of the upper section 31 of the aligner 30 to above the maxillary bone screw 20 to protect the soft tissue of the lip from irritation by the bone screw 20 and elastic 25. For example, the extension 38 can be a paddle-shaped member made of acrylic with an internal wire reinforcement soldered or welded to the upper labial-buccal archwire 33. The extension 38 should preferably have a sufficient thickness to lift the soft tissue of the lip away from excessive contact with the head of the bone screw 20 and elastic 25.

Returning to the embodiment of the aligner 30 shown in FIGS. 1 and 3, it should be noted that the forces exerted by the elastics 25 may tend to pull the lower section 32 of the aligner forward and out of contact with the lower teeth. The lower section 32 can include a posterior flange or surface as shown in FIG. 3 that extends around the distal aspect of the last tooth to provide additional retention.

The aligner 30 can also be equipped with a number of ball clasps 56 (shown in FIG. 11) or fingers that extend into the interproximal spaces between the patient's posterior teeth to removably secure the aligner 30 in place. A ball clasp is a wire with a ball on its end that extends into the embrasure between adjacent teeth for retention. The aligner could also use a series of interproximal wires for retention. All of these should be considered to be “ball clasps” for the purposes of this disclosure.

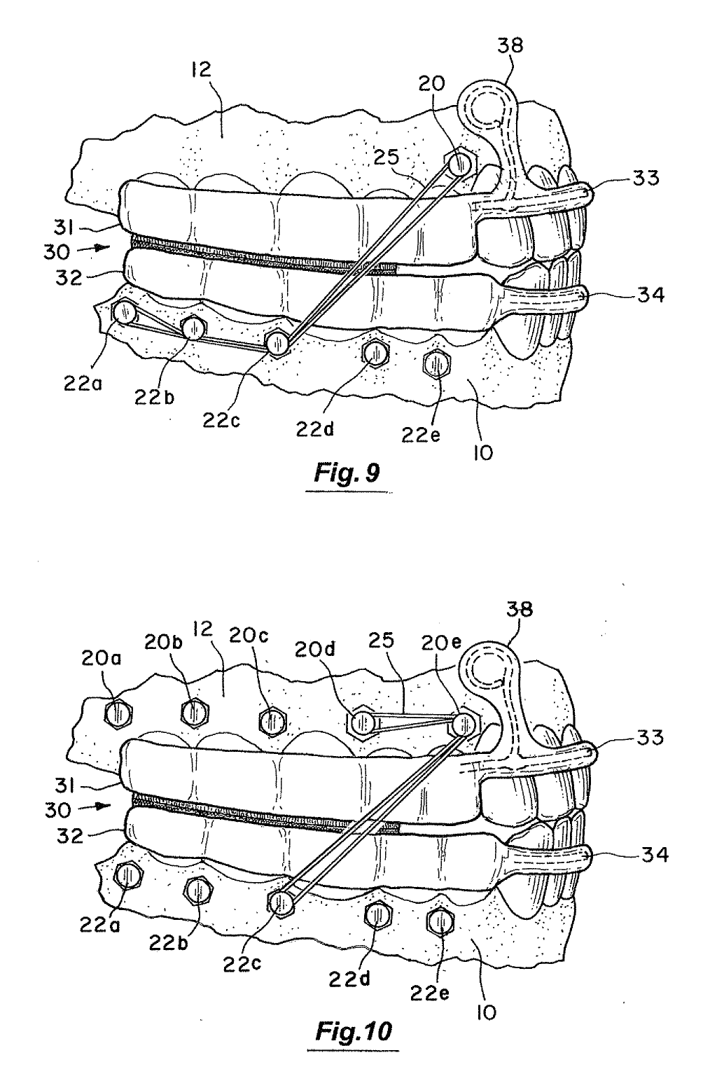

FIG. 8 is a side view of a patient's dental anatomy showing an implementation of the present invention using multiple mandibular bone screws 22a-22d. This configuration can be used to allow multiple elastics 25 to be stretched between the maxilla 12 and mandible 10, and/or to change the angle of the force exerted by an elastic. FIG. 9 is a side view of a patient's dental anatomy showing an alternative arrangement for attaching an elastic 25 using multiple mandibular bone screws 22a-22e. FIG. 10 is a side view of showing another arrangement for attaching an elastic 25 using multiple maxillary bone screws 20a-20e and mandibular bone screws 22a-22e. This configuration allows the elastics 25 to exert greater force biasing the patient's jaw toward a closed position and also allows a greater range of motion.

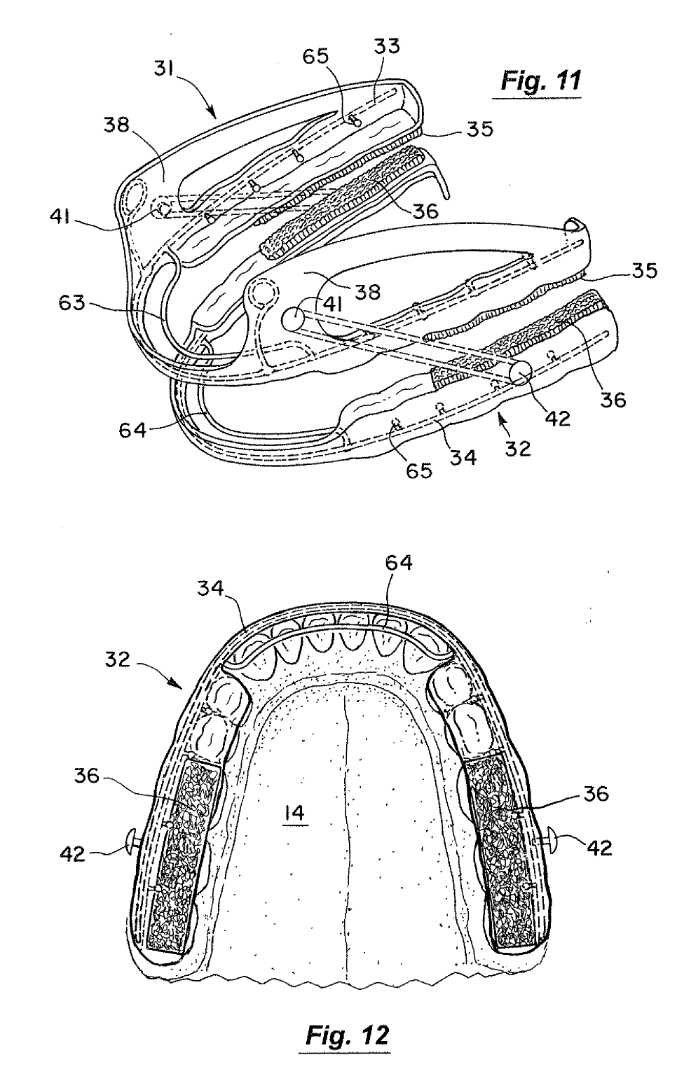

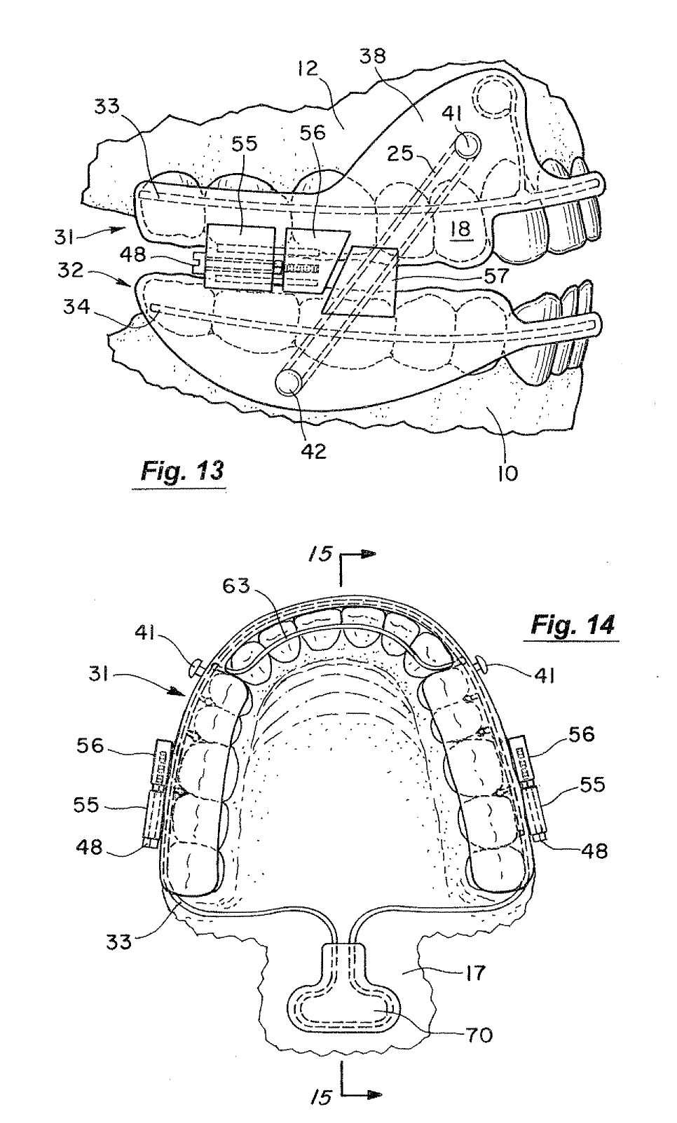

FIGS. 11-13 show another embodiment of the aligner 30. FIG. 11 is an exploded perspective view of the upper and lower sections 31, 32 of this embodiment of the aligner 30. FIG. 12 is a top view of the lower section 32 of the aligner 30 illustrated in FIG. 11 on a patient's lower teeth. FIG. 13 is a side view of the embodiment of the aligner 30 illustrated in FIG. 11 with a screw adjustment mechanism allowing a range of anterior-posterior movement between the upper and lower sections 31, 32 of the aligner 30. This embodiment includes embedded labial-buccal archwires 33, 34 and a screw adjustment mechanism, as previously discussed. However, the upper and lower segments 31, 32 of the aligner 30 also include lingual wires 63, 64 to help retain the aligner 30 in place.

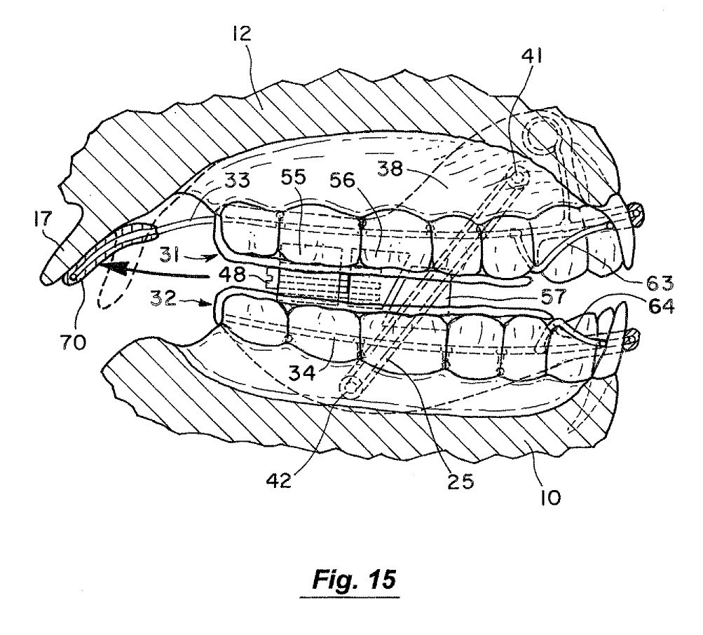

FIGS. 14 and 15 show another embodiment of the aligner 30 incorporating a soft palate extension 70 extending posteriorly from the upper segment 31 of the aligner 30 to elevate and tighten the patient's soft palate tissue 17. FIG. 14 is a bottom view of an embodiment of the upper section 31 of an aligner incorporating the posterior extension 70. FIG. 15 is a side cross-sectional view showing the manner in which the soft tissue 17 of the patients palate is elevated by the posterior extension 70. In this embodiment, the soft palate extension 70 is a substantially planar polymeric paddle with a wire embedded in the polymer. The wire can be continuation of the upper labial-buccal archwire 33. This wire can be readily deformed to achieve a desired position and orientation for the paddle relative to a patient's palate. Its shape and contour can also be adjusted to elevate and tighten the soft palate.

The embodiments of the aligner 30 shown in FIGS. 11-15 include a number of buttons 41, 42 on the labial-buccal aspects of the upper and lower segments 31, 32 of the aligner 30 for engaging elastics 25. These buttons 41, 42 can be used alone or in combination with bone screws 20, 22 to attach elastics 25 in any desired arrangement. In addition, it may be possible to entirely omit either the upper section 31 or the lower section 32 of the aligner 30, if bone screws 20, 22 are attached to either the maxilla or mandible to anchor one end of the elastics 25.

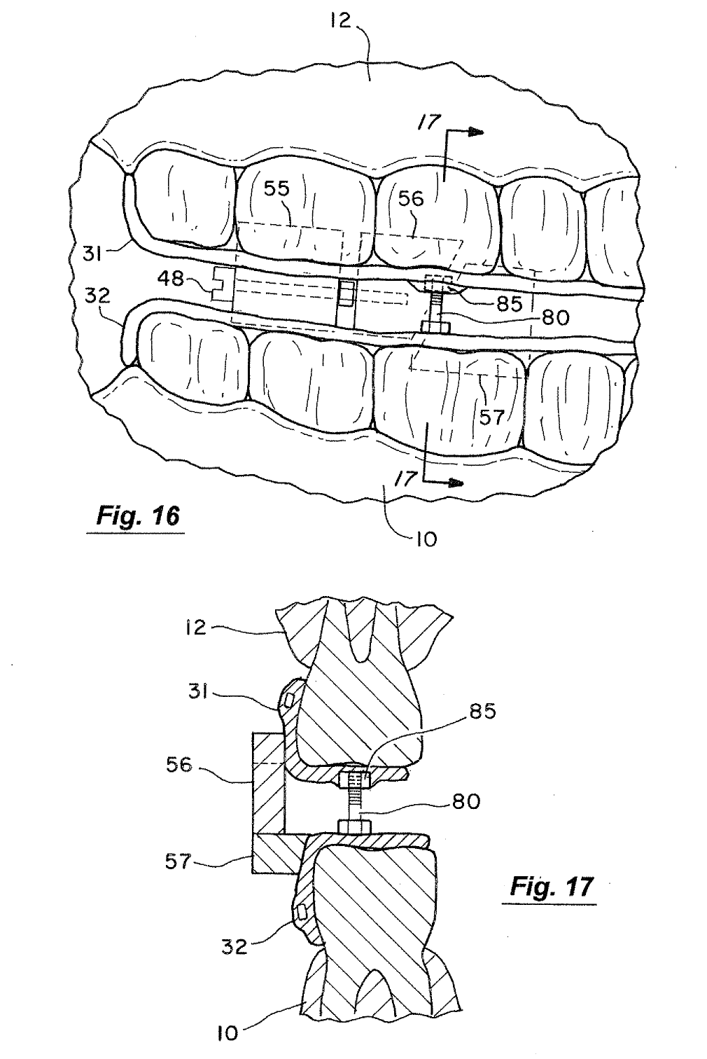

FIG. 16 is a vertical cross-sectional view showing another embodiment of an aligner that includes a screw 80 for adjusting the minimum vertical separation between the upper and lower sections 31, 32 of the aligner. FIG. 17 is an orthogonal vertical cross-sectional view of the embodiment depicted in FIG. 16. In these drawings, the screw 80 is threaded into a nut 85 embedded into the occlusal surface of the upper section 31 of the aligner. Preferably, the screw 80 is placed in vertical alignment with the central fossa of the mandibular first molar Alternatively, the screw could be threaded into a nut embedded into the occlusal surface of the lower section 32 of the aligner.

The above disclosure sets forth a number of embodiments of the present invention described in detail with respect to the accompanying drawings. Those skilled in this art will appreciate that various changes, modifications, other structural arrangements, and other embodiments could be practiced under the teachings of the present invention without departing from the scope of this invention as set forth in the following claims.

I claim:

1. An aligner for placement between a patient's upper and lower teeth to maintain a desired positioning between a patient's maxilla and mandible, said aligner comprising;
an upper section to receive the patient's upper teeth having:
(a) a polymeric body extending on the labial-buccal and occlusal aspects of the upper teeth, without extending on the lingual aspects of the upper teeth; and
(b) a labial-buccal archwire embedded within the polymeric body;
a lower section to receive the patient's lower teeth having:
(a) a polymeric body extending on the labial-buccal and occlusal aspects of the lower teeth, without extending on the lingual aspects of the lower teeth; and
(b) a labial-buccal archwire embedded within the polymeric body; and
an adjustment mechanism removably securing the upper section to the lower section and providing a degree of adjustability along an anterior-posterior axis between the upper section and the lower section.

2. The aligner of claim 1 wherein the adjustment mechanism comprises complementary patches of hook-and-loop fastener material on the occlusal surfaces of the upper section and lower section.

3. The aligner of claim 1 wherein the adjustment mechanism comprises:
a first block secured to the occlusal surface of the upper section;
a second block secured to the occlusal surface of the lower section; and
an adjustment screw to adjust the spacing between the first block and the second block.

4. The aligner of claim 1 further comprising a posterior extension extending posteriorly from the upper section to elevate the patient's soft palate tissue.

5. The aligner of claim 1 further comprising an extension extending upward on the labial aspect of the maxillary anterior portion of the upper section.

6. The aligner of claim 1 wherein the polymeric body of the upper section extends only on the posterior teeth.

7. The aligner of claim 1 wherein the polymeric body of the lower section extends only on the posterior teeth.

8. The aligner of claim 1 further comprising at least one button on the labial-buccal aspects of the aligner for engaging an elastic.

9. The aligner of claim 1 further comprising at least one ball clasp for removably securing the aligner to a patient's teeth.

10. An aligner for placement between a patient's upper and lower teeth to maintain a desired positioning between a patient's maxilla and mandible, said aligner comprising:
an upper section to receive the patient's upper teeth having:
(a) a polymeric body extending on the labial-buccal and occlusal aspects of the upper posterior teeth, without extending on the lingual aspects of the upper teeth; and
(b) a labial-buccal archwire having a posterior portion embedded within the polymeric body and an anterior portion extending around the labial aspect of the upper anterior teeth;
a lower section to receive the patient's lower teeth having:
(a) a polymeric body extending on the labial-buccal and occlusal aspects of the lower posterior teeth, without extending on the lingual aspects of the lower teeth; and
(b) a labial-buccal archwire having a posterior portion embedded within the polymeric body and an anterior portion extending around the labial aspect of the lower anterior teeth; and
an adjustment mechanism removably securing the upper section to the lower section and providing a degree of adjustability along an anterior-posterior axis between the upper section and the lower section.

11. The aligner of claim 10 wherein the adjustment mechanism comprises complementary patches of hook-and-loop fastener material on the occlusal surfaces of the upper section and lower section.

12. The aligner of claim 10 wherein the adjustment mechanism comprises:
a first block secured to the occlusal surface of the upper section;
a second block secured to the occlusal surface of the lower section; and
an adjustment screw to adjust the spacing between the first block and the second block.

13. The aligner of claim 10 further comprising a posterior extension extending posteriorly from the upper section to elevate the patient's soft palate tissue.

14. The aligner of claim 10 further comprising an extension extending upward on the labial aspect of the maxillary anterior portion of the upper section.

15. The aligner of claim 10 further comprising at least one button on the labial-buccal aspects of the aligner for engaging an elastic.

16. The aligner of claim 10 further comprising at least one ball clasp for removably securing the aligner to a patient's teeth.
